(19) United States
(12) Patent Application Publication
(10) Pub. No.: US 2011/0238164 A1
(43) Pub. Date: Sep. 29, 2011

(54) Device and Method for Placing and Positioning a Heart Valve

(76) Inventors: Andrzej M. Malewicz, Minneapolis, MN (US); Alan Stewart, New York, NY (US)

(21) Appl. No.: 12/892,205

(22) Filed: Sep. 28, 2010

Related U.S. Application Data
(60) Provisional application No. 61/246,832, filed on Sep. 29, 2009.

(51) Int. Cl. A61F 2/24; A61B 17/00

(52) U.S. Cl. 623/2.11; 606/1

(57) ABSTRACT

A positioning tool comprises an elongate positioning member having a first end and a second end, a tubular positioning body coupled to the first end of the elongate positioning member and including a first end, a second end, and an attachment structure disposed within an open center of the positioning body, and a plurality of pairs of alignment apertures, each pair of alignment apertures including a first alignment aperture adjacent to the first end of the positioning body and a second alignment aperture adjacent to the second end of the positioning body.

### CROSS-REFERENCE TO RELATED APPLICATIONS

This application claims priority under 35 U.S.C. §119(e)(1) to U.S. Provisional Patent Application Ser. No. 61/246,832, filed Sep. 29, 2009, entitled “Device and Method for Placing and Positioning a Heart Valve”, the entire teachings of which are incorporated herein by reference.

### FIELD OF THE INVENTION

The present invention relates to replacement heart valves. More particularly, the present invention relates to a device and method for placing and positioning a heart valve within a patient.

### DESCRIPTION OF THE RELATED ART

The transport of vital fluids in the human body is largely regulated by valves. Physiological valves are designed to prevent the backflow of bodily fluids, such as blood, lymph, urine, bile, etc., thereby keeping the body's fluid dynamics unidirectional for proper homeostasis. For example, venous valves maintain the upward flow of blood, particularly from the lower extremities, back toward the heart, while lymphatic valves prevent the backflow of lymph within the lymph vessels, particularly those of the limbs.

Because of their common function, valves share certain anatomical features despite variations in relative size. The cardiac valves are among the largest valves in the body with diameters that may exceed 30 mm, while valves of the smaller veins may have diameters no larger than a fraction of a millimeter. Regardless of their size, however, many physiological valves are situated in specialized anatomical structures known as sinuses. Valve sinuses can be described as dilations or bulges in the vessel wall that house the valve. The geometry of the sinus has a function in the operation and fluid dynamics of the valve. One function is to guide fluid flow so as to create eddy currents that prevent the valve leaflets from adhering to the wall of the vessel at the peak of flow velocity, such as during systole. Another function of the sinus geometry is to generate currents that facilitate the precise closing of the leaflets at the beginning of backflow pressure. The sinus geometry is also important in reducing the stress exerted by differential fluid flow pressure on the valve leaflets or cusps as they open and close.

Thus, for example, the eddy currents occurring within the sinuses of Valsalva in the natural aortic root have been shown to be important in creating smooth, gradual and gentle closure of the aortic valve at the end of systole. Blood is permitted to travel along the curved contour of the sinus and onto the valve leaflets to effect their closure, thereby reducing the pressure that would otherwise be exerted by direct fluid flow onto the valve leaflets. The sinuses of Valsalva also contain the coronary ostia, which are outflow openings of the arteries that feed the heart muscle. When valve sinuses contain such outflow openings, they serve the additional purpose of providing blood flow to such vessels throughout the cardiac cycle.

When valves exhibit abnormal anatomy and function as a result of valve disease or injury, the unidirectional flow of the physiological fluid they are designed to regulate is disrupted, resulting in increased hydrostatic pressure. For example, venous valvular dysfunction leads to blood flowing back and pooling in the lower legs, resulting in pain, swelling and edema, changes in skin color, and skin ulcerations that can be extremely difficult to treat. Lymphatic valve insufficiency can result in lymphedema with tissue fibrosis and gross distention of the affected body part. Cardiac valvular disease may lead to pulmonary hypertension and edema, atrial fibrillation, and right heart failure in the case of mitral and tricuspid valve stenosis; or pulmonary congestion, left ventricular contractile impairment and congestive heart failure in the case of mitral regurgitation and aortic stenosis. Regardless of their etiology, all valvular diseases result in either stenosis, in which the valve does not open properly, impeding fluid flow across it and causing a rise in fluid pressure, or insufficiency/regurgitation, in which the valve does not close properly and the fluid leaks back across the valve, creating backflow. Some valves are afflicted with both stenosis and insufficiency, in which case the valve neither opens fully nor closes completely.

Because of the potential severity of the clinical consequences of valve disease, numerous surgical techniques may be used to repair a diseased or damaged heart valve. For example, these surgical techniques may include annuloplasty (contracting the valve annulus), quadrangular resection (narrowing the valve leaflets), commissurotomy (cutting the valve commissures to separate the valve leaflets), or decalcification of valve and annulus tissue. Alternatively, the diseased heart valve may be replaced by a prosthetic valve. Where replacement of a heart valve is indicated, the dysfunctional valve is typically removed and replaced with either a mechanical or tissue valve.

When a diseased heart valve is replaced with a mechanical or tissue valve, it is critical that the replacement valve be positioned properly within the sinus area. For example, care must be taken so that the inflow end of the replacement valve is positioned within the proper plane adjacent the inflow annulus, the commissural tabs on the replacement valve are positioned at the proper height with respect to the inflow annulus, and the outflow end of the valve is not angularly misaligned or “twisted” with respect to the inflow end. However, positioning a replacement valve in such a manner has proven difficult for surgeons in the field.

Thus, what is needed is an improved system and method for the proper positioning of a replacement valve at a target site within a patient.

### SUMMARY OF THE INVENTION

The present invention solves the foregoing problems by providing a positioning tool comprising an elongate positioning member having a first end and a second end, a tubular positioning body coupled to the first end of the elongate positioning member and including a first end, a second end, and an attachment structure disposed within an open center of the positioning body, and a plurality of pairs of alignment apertures, each pair of alignment apertures including a first alignment aperture adjacent to the first end of the positioning body and a second alignment aperture adjacent to the second end, of the positioning body.

In another aspect of the present invention, a method for positioning a heart valve within a patient comprises providing a positioning tool including an elongate positioning member, a positioning body disposed at an end of the elongate positioning member, and a plurality of pairs of alignment apertures formed in the positioning body, each pair of alignment apertures including spaced apart first and second alignment apertures. The method further comprises sliding the positioning body into an aortic sinus region until the second alignment apertures are positioned adjacent to an inflow annulus of the sinus region, aligning each of the second alignment apertures with an end of a commissure adjacent to the inflow annulus, marking a first commissure location of each of the second alignment apertures, retracting the positioning body until the first alignment apertures are substantially coplanar with the inflow annulus and aligned with the marked first commissure locations, and marking a second commissure location of each of the second alignment apertures.

### BRIEF DESCRIPTION OF THE DRAWINGS

FIGS. 1A and 1B illustrate an exemplary valve during normal operation. FIG. 1A illustrates the valve in an open position during peak flow, and FIG. 1B illustrates the valve in a closed position to prevent backflow of the fluid across the valve.

FIG. 2A is a top view illustrating the anatomy of a typical aortic valve.

FIG. 2B is a cross-sectional view of the aortic valve of FIG. 2A.

FIG. 2C is a perspective view of the aortic valve of FIG. 2A showing the inflow end, outflow end, and commissural posts in phantom lines.

FIG. 3 is a schematic representation of the geometry and relative dimensions of the valve sinus region of an aorta.

FIG. 4 is an exemplary bioprosthetic valve that may be used in an aortic valve replacement procedure.

FIG. 5 depicts an exemplary embodiment of the bioprosthetic valve of FIG. 4 positioned within an aorta.

FIG. 6 is a side view of one exemplary embodiment of a positioning tool in accordance with the present invention.

FIG. 7 is a perspective view of a portion of the positioning tool of FIG. 6.

FIG. 8 is a diagram illustrating the first step of an exemplary valve positioning method wherein a positioning body of the positioning tool is positioned within an aortic sinus region.

FIGS. 9A, 9B, and 9C are diagrams illustrating a first marking step in accordance with the method of the present invention.

FIG. 10 is a diagram illustrating the positioning body in a retracted position in accordance with the method of the present invention.

FIGS. 11A and 11B are diagrams illustrating alternative forms of a second marking step in accordance with the method of the present invention.

### DETAILED DESCRIPTION OF THE INVENTION

While this invention may be embodied in many different forms, there are described in detail herein various embodiments of the invention. This description is an exemplification of the principles of the invention and is not intended to limit the invention to the particular embodiments illustrated.

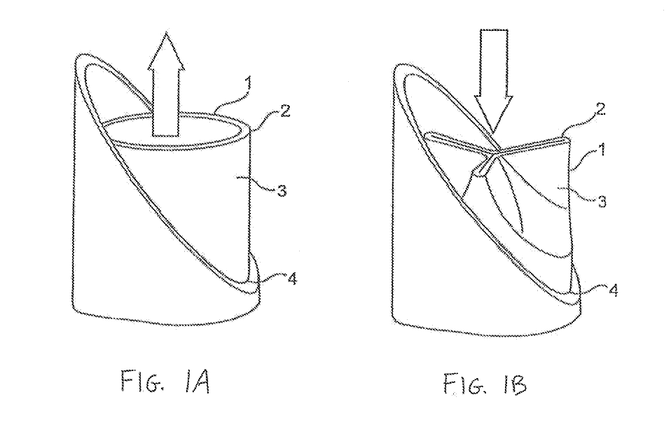

Turning now to the Figures, the present invention relates to a device and method for placing and positioning a heart valve within a patient. FIGS. 1A and 1B generally illustrate one exemplary embodiment of a heart valve 1. As illustrated in FIGS. 1A and 1B, valve 1 includes a distal outflow end 2, a plurality of leaflets 3, and a proximal inflow end 4. A typical valve functions similar to a collapsible tube in that it opens widely during systole or in response to muscular contraction to enable unobstructed forward flow across the valvular orifice, as illustrated in FIG. 1A. In contrast, as forward flow decelerates at the end of systole or contraction, the walls of the tube are forced centrally between the sites of attachment to the vessel wall and the valve closes completely as illustrated in FIG. 1B.

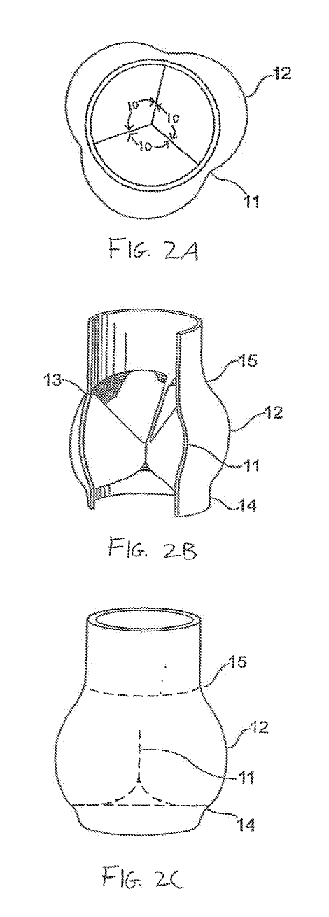

FIGS. 2A, 2B, and 2C illustrate the anatomy of a typical aortic root having an aortic valve therein. In particular, FIG. 2A shows a top view of a closed valve with three valve sinuses 12, FIG. 2B shows a perspective sectional view of the closed valve, and FIG. 2C shows a view from outside the vessel wall.

One important factor in the positioning of a replacement valve is the architecture of the valve sinuses. Valve sinuses are dilations of the vessel wall that surround the natural valve leaflets. Typically in the aortic valve, each natural valve leaflet has a separate sinus bulge 12 or cavity that allows for maximal opening of the leaflet at, peak flow without permitting contact between the leaflet and the vessel wall. As illustrated in FIGS. 2A, 2B, and 2C, the extent of each sinus 12 within the aortic root is generally defined by the commissures 11, vessel wall 13, inflow end 14, and outflow end 15. The proximal intersection between the sinus cavities defines the commissures 11. In a typical tri-leaflet valve as illustrated in FIG. 2A, adjacent commisures 11 are spaced by an angle 10 of about 120 degrees.

FIGS. 2B and 2C also show the narrowing diameter of the sinuses at both the inflow end 14 and the outflow end 15, thereby forming the annulus and sinotubular junction, respectively, of the sinus region. Thus, the valve sinuses form a natural compartment to support the operation of the valve by preventing contact between the leaflets and the vessel wall, which, in turn, may lead to adherence of the leaflets and/or result in detrimental wear and tear of the leaflets. The valve sinuses are also designed to share the stress conditions imposed on the valve leaflets during closure when fluid pressure on the closed leaflets is greatest. The valve sinuses further create favorable fluid dynamics through currents that soften an otherwise abrupt closure of the leaflets under conditions of high backflow pressure. Lastly, the sinuses ensure constant flow to any vessels located within the sinus cavities.

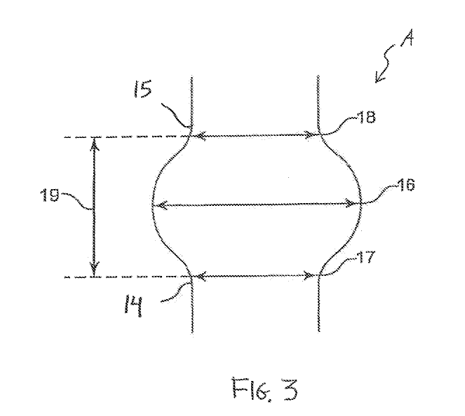

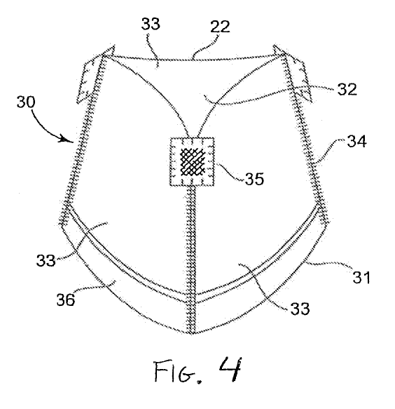

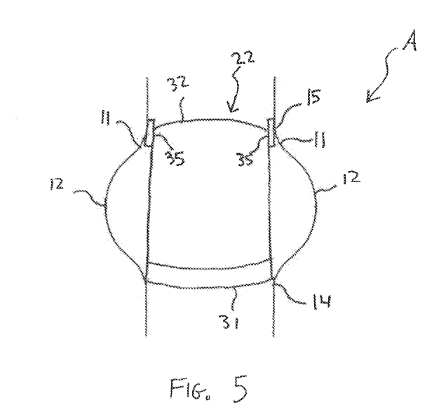

FIG. 3 is a schematic representation of the geometry and relative dimensions of the valve sinus region of an aorta A. As shown in FIG. 3, the valve sinus region is characterized by certain relative dimensions which remain substantially constant regardless of the actual size of the sinuses. Generally, the diameter of the sinus is at its largest at the center of the sinus cavities 16, while there is pronounced narrowing of the sinus region at both the inflow annulus 17 near the inflow end 14 and the outflow sinotubular junction 18 near the outflow end 15. Furthermore, the height of the sinus 19 (i.e. the distance between the inflow annulus 17 and the outflow sinotubular junction 18) remains substantially proportional to its overall dimensions. It is thus apparent that the sinus region forms an anatomical compartment with certain substantially constant features that are uniquely adapted to house a valve. The device and method of the present invention are designed to utilize these known anatomical features of the native sinus region for optimal placement and positioning of a replacement heart valve.

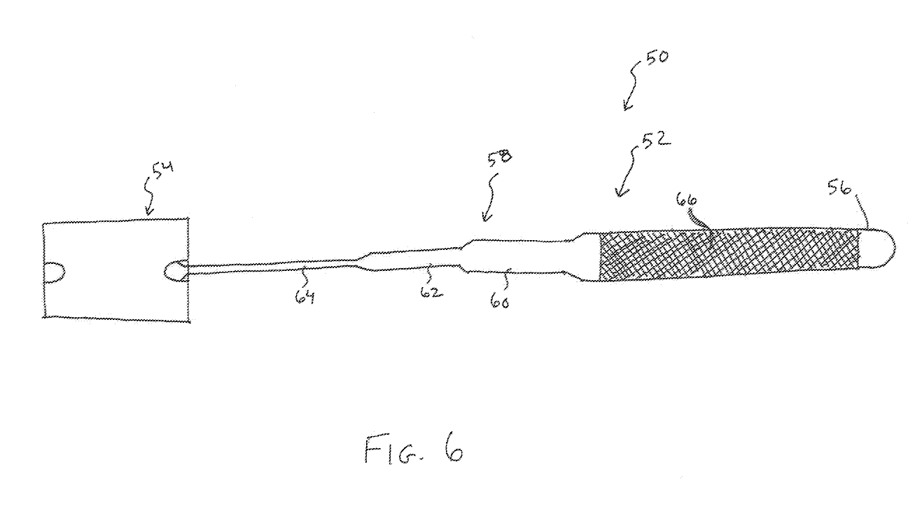

FIG. 4 is a perspective view of a replacement valve 22, which represents one exemplary embodiment of a typical, tri-leaflet replacement valve that may be used in an aortic valve replacement procedure. The replacement valve 22 includes a valve body 30 having a proximal inflow end 31 and a distal outflow end 32. The valve body 30 includes a plurality of valve tissue leaflets 33 joined by seams 34, wherein each seam 34 is formed by a junction of two leaflets 33. A commissural tab 35 extends from each seam 34 at the distal end of the valve body 30. The inflow end 31 of the valve body 30 includes a peripheral edge that may be scalloped or straight. In addition, the inflow end 31 of the valve body 30 may further comprise reinforcement structure 36 that may be stitched or otherwise attached thereto.

The valve leaflets 33 may be constructed of any suitable material, including but not limited to expanded polytetrafluoroethylene (ePTFE), equine pericardium, bovine pericardium, or native porcine valve leaflets similar to currently available bioprosthetic aortic valves. Other materials may prove suitable as will be appreciated by those skilled in the art.

The number of commissural tabs 35 generally ranges from two to four, depending on the number of commissural posts present in the valve sinus. In the exemplary embodiment illustrated in FIG. 4, the tri-leaflet replacement valve 22 includes three distinct commissural tabs 35 for placement within a sinus that features three natural commissural posts, such as the one illustrated in FIGS. 2A, 2B, and 2C.

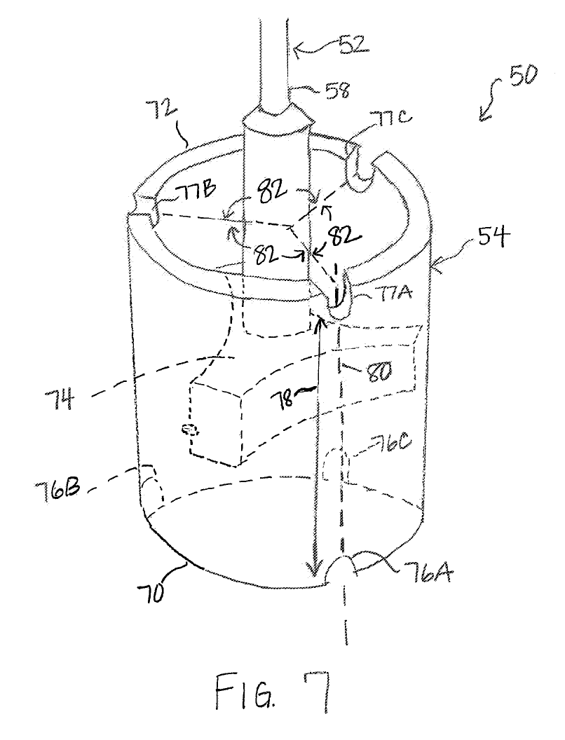

FIG. 5 is a section view of the replacement valve 22 positioned within the aorta A of FIG. 3. As illustrated in FIG. 5, two of the commissural tabs 35 are illustrated aligned with corresponding commissures 11 of the valve sinus 12 (the third commissural tab 35 being omitted for purposes of illustration). As appreciated by those skilled in the art, when positioning the valve 22 within the aorta A it is important to ensure that there is approximately 120 degrees of separation between adjacent commissural tabs 35 so as to match-up and align with the natural spacing of the commissures 11. It is also important to ensure that the inflow end 31 of the valve 22 is not angularly misaligned or “twisted” relative to the outflow end 32. Furthermore, it is important to ensure that the valve 22 is positioned such that the height of the commissural tabs 35 relative to the inflow end 14 substantially matches the height of the valve sinus 19. These objects are addressed by the device and method for placing and positioning a heart valve in accordance with the present invention.

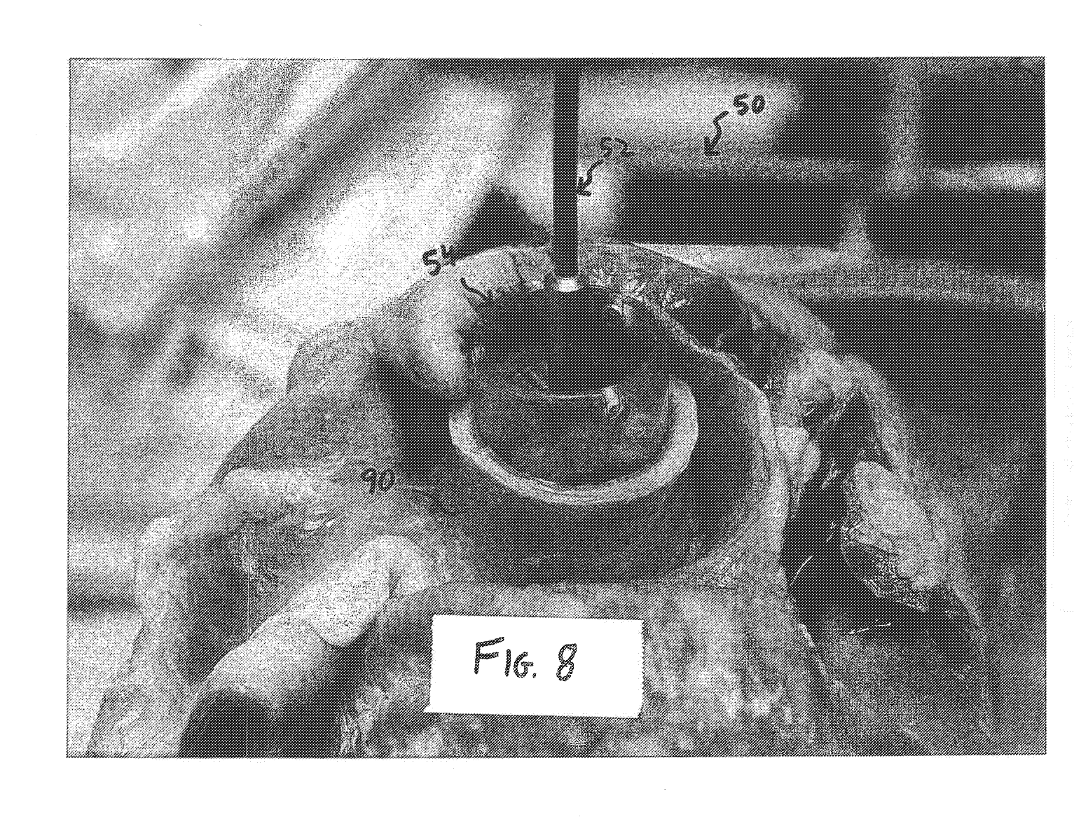

FIG. 6 is a side view of one exemplary positioning tool 50 in accordance with the present invention. The positioning tool 50 generally includes an elongate positioning member 52 and a positioning body 54 coupled thereto. The positioning member 52 generally includes a handle 56 and a shaft 58 including a first portion 60, a second portion 62, and a third portion 64. The handle 56 may include gripping means 66 structured to allow a surgeon to maintain a firm grip on the handle 56 while utilizing the positioning tool 50.

As illustrated in FIG. 6, the first portion 60, second portion 62, and third portion 64 of the shaft 58 have diameters that differ from one another. Particularly, the diameters decrease from the first portion 60 to the third portion 64. However, the shape and structure of the positioning member 52 may vary without departing from the intended scope of the present invention. In one alternative embodiment the shaft 58 may have a substantially uniform diameter. Furthermore, the diameter of the shaft 58 may be substantially similar to that of the handle 56 such that the positioning member 52 is structured as an elongate member with a substantially uniform diameter. Numerous other configurations are also contemplated.

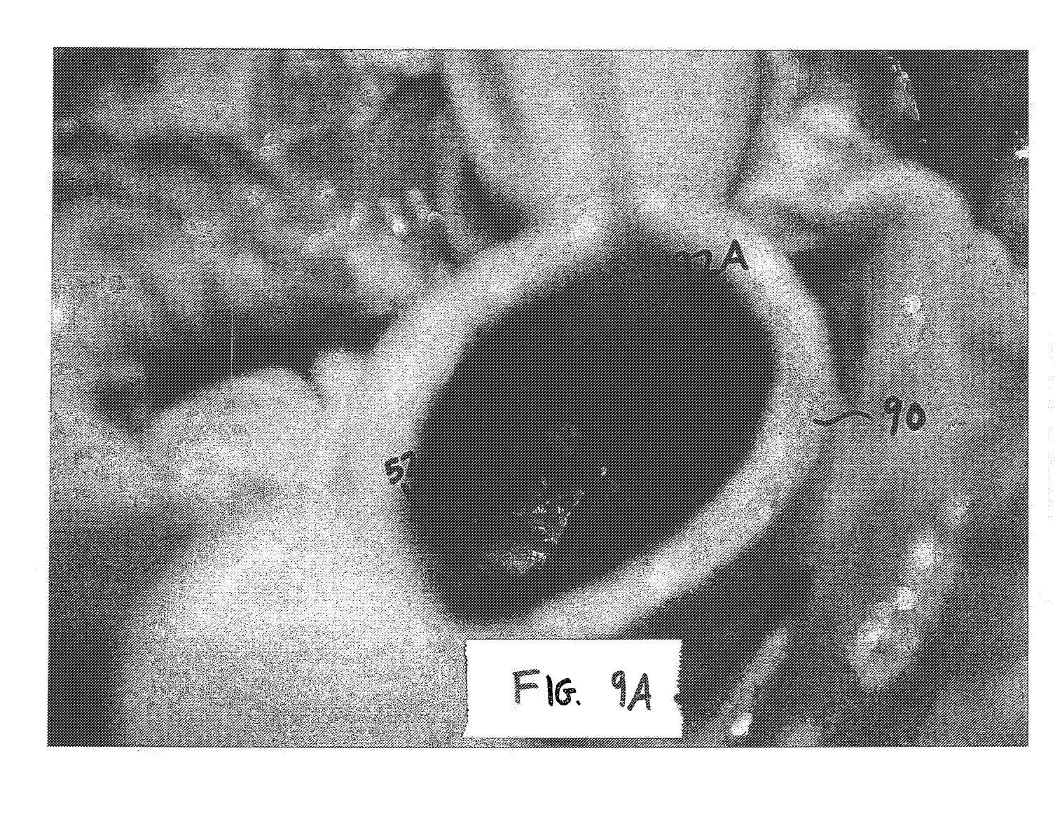

FIG. 7 is a perspective view of a portion of the positioning tool 50 illustrating various details of the positioning body 54. As illustrated in FIG. 7, the positioning body 54 includes a first end 70, a second end 72, and an attachment structure 74 positioned within an open center of the positioning body 54 that is structured for coupling to the shaft 58 of the positioning member 52. In one exemplary embodiment the attachment structure 74 may be structured to threadably receive and engage the shaft 58 in order to releasably couple the components together. However, any suitable attachment means may be used without departing from the intended scope of the present invention including, but not limited to, adhesives, heat welding, or compression fit. Thus, the shaft 58 may be releasably or permanently coupled to the positioning body 54.

As illustrated in FIG. 7, the positioning body 54 includes a first pair of alignment notches 76A and 77A, a second pair of alignment notches 76B and 77B, and a third pair of alignment notches 76C and 77C. Although the present disclosure describes these three pairs of structural features as “notches,” any structural feature that extends through the outer wall of the positioning body 54, including but not limited to a “slot” or an “aperture,” is contemplated and within the intended scope of the present invention. The distance 78 between each of the notches in a pair is substantially equivalent to the typical height of an aortic sinus. Each of the notches in a pair is positioned along a straight line 80 that is substantially perpendicular to the first and second ends 70 and 72 of the positioning body 54 such that the notches are angularly aligned. Furthermore, adjacent pairs of alignment notches are separated by an angle 82 of about 120 degrees. As will be illustrated in the following Figures, these features of the positioning body 54 form a template for the proper placement and positioning of a replacement valve and help to ensure that the valve is positioned such that the height of the replacement valve substantially matches the height of the valve sinus, that the inflow end of the valve is not twisted relative to the outflow end, and that there is approximately 120 degrees of separation between adjacent commissural tabs so as to match-up and align with the spacing of the natural commissures.

The various components of the positioning tool 50 may be manufactured using any suitable material. In one exemplary embodiment the positioning member 52 may be manufactured with a surgical-grade stainless steel and the positioning body 54 may be manufactured with a biocompatible plastic. However, numerous other materials may be used as will be appreciated by those skilled in the art.

Now that the general structure of one exemplary positioning tool 50 has been set forth in detail, an exemplary method of positioning a valve with a positioning tool in accordance with the present invention will be described. Although the method will be described with reference to the positioning tool 50, those skilled in the art will appreciate that the inventive method may be practiced with alternative positioning tools without departing from the intended scope of the present invention.

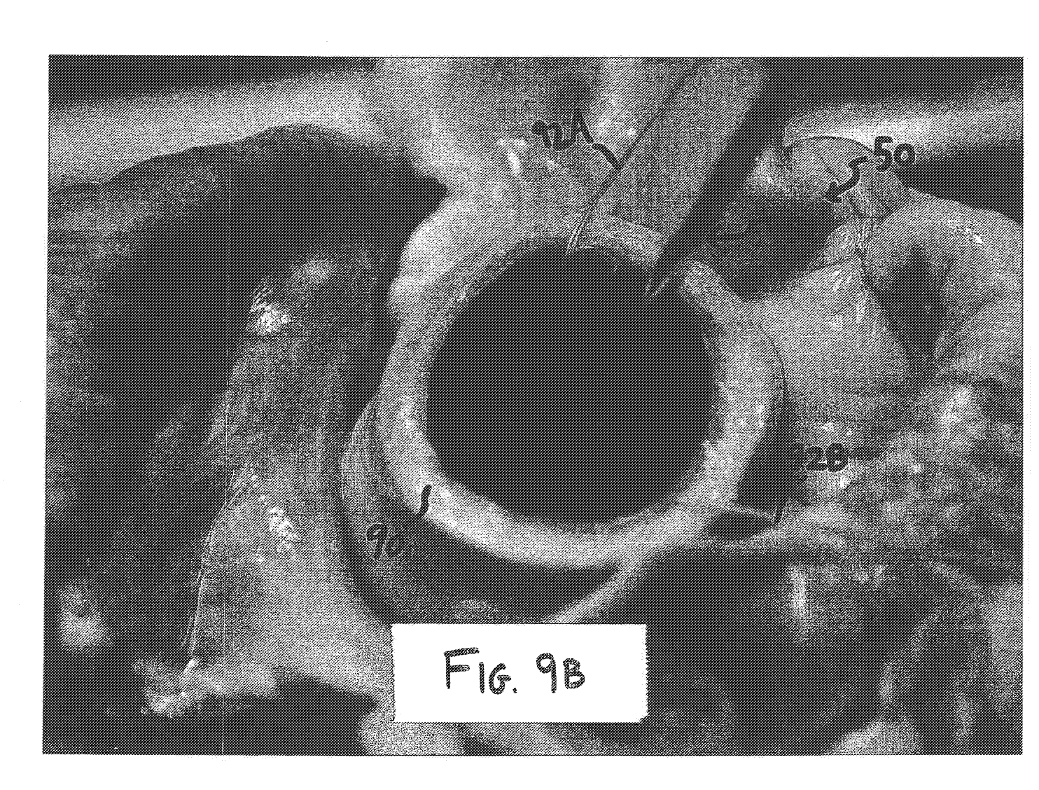

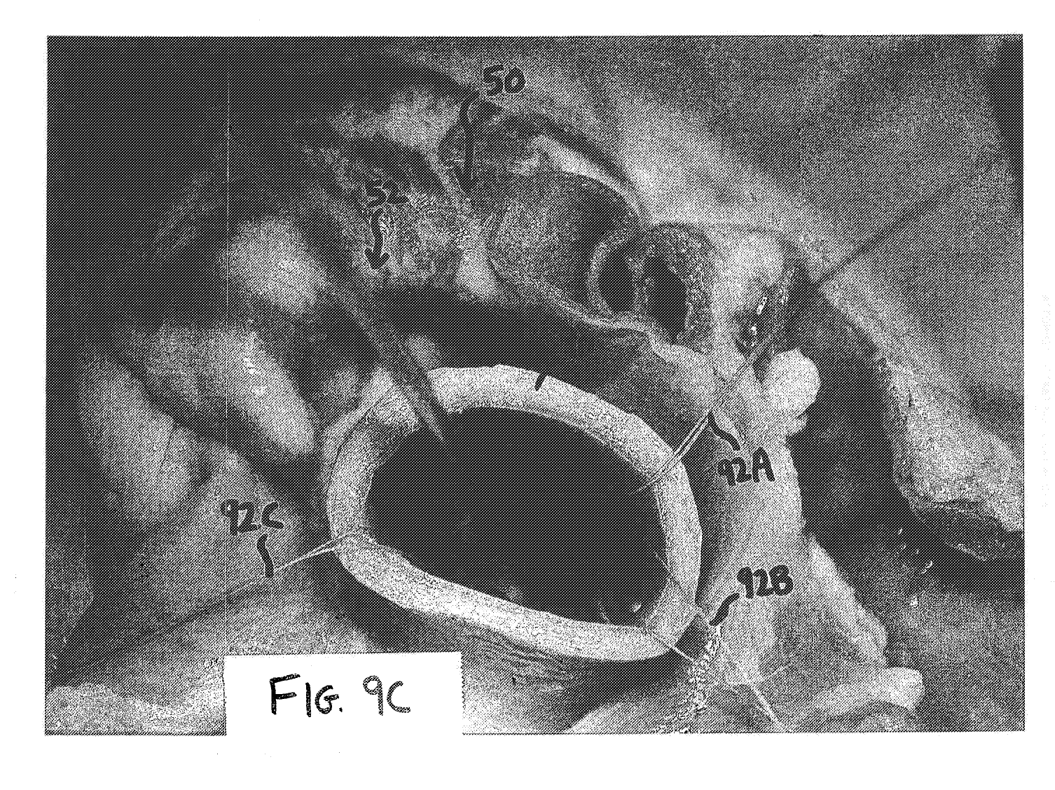

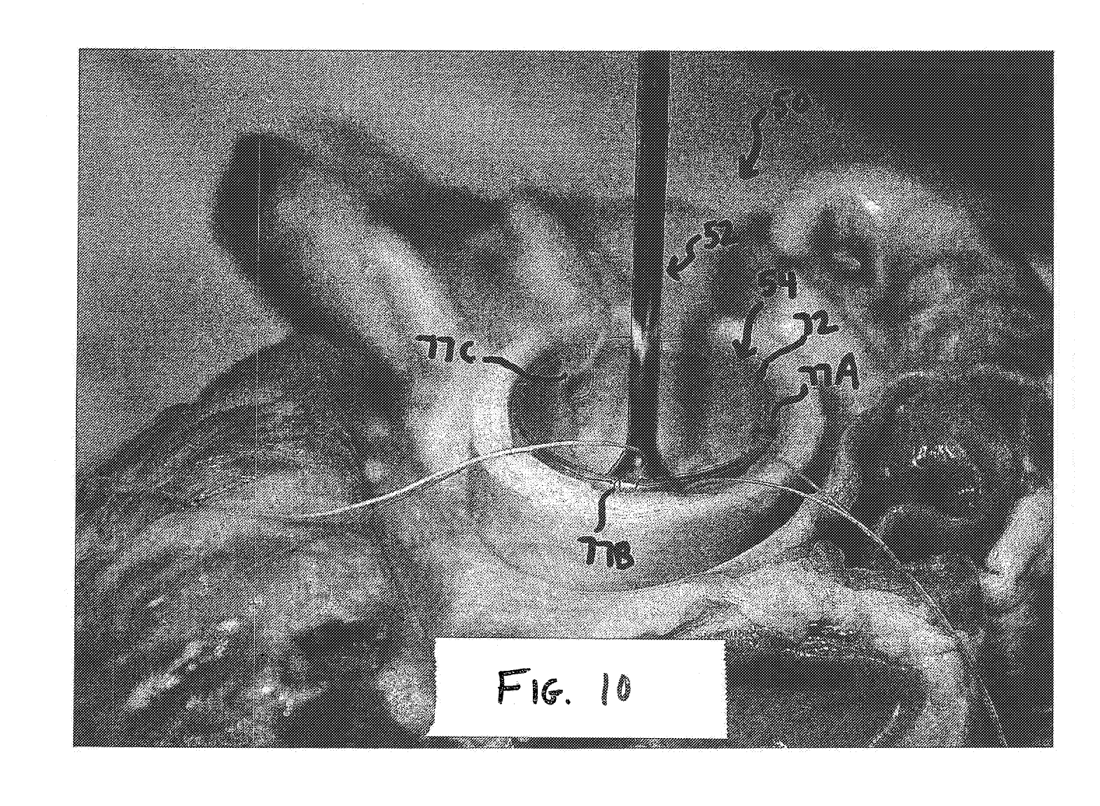

As illustrated in FIG. 8, the process begins by aligning the positioning body 54 of the positioning tool 50 with an aortic sinus region 90. The surgeon then slides the positioning body 54 into the aortic sinus region 90 until the alignment notches 77A, 77B, and 77C on the second end 72 of the positioning body 54 are positioned adjacent to the inflow annulus of the sinus region 90. The positioning body 54 is then rotated until the alignment notches 77A, 77B, and 77C each align with the lower end of one of the commissures within the sinus region 90. Once the alignment notches 77A, 77B, and 77C are properly aligned, the locations of the notches are marked using any suitable marking means as will be appreciated by those skilled in the art. In one exemplary embodiment as illustrated herein, the locations of the alignment notches (which correspond to the positions of the commissures) may be marked with a suture. Particularly, FIG. 9A illustrates the step of marking the position of alignment notch 77A with a first suture 92A, FIG. 9B illustrates the step of marking the position of alignment notch 77B with a second suture 92B, and FIG. 9C illustrates the step of marking the position of alignment notch 77C with a third suture 92C.

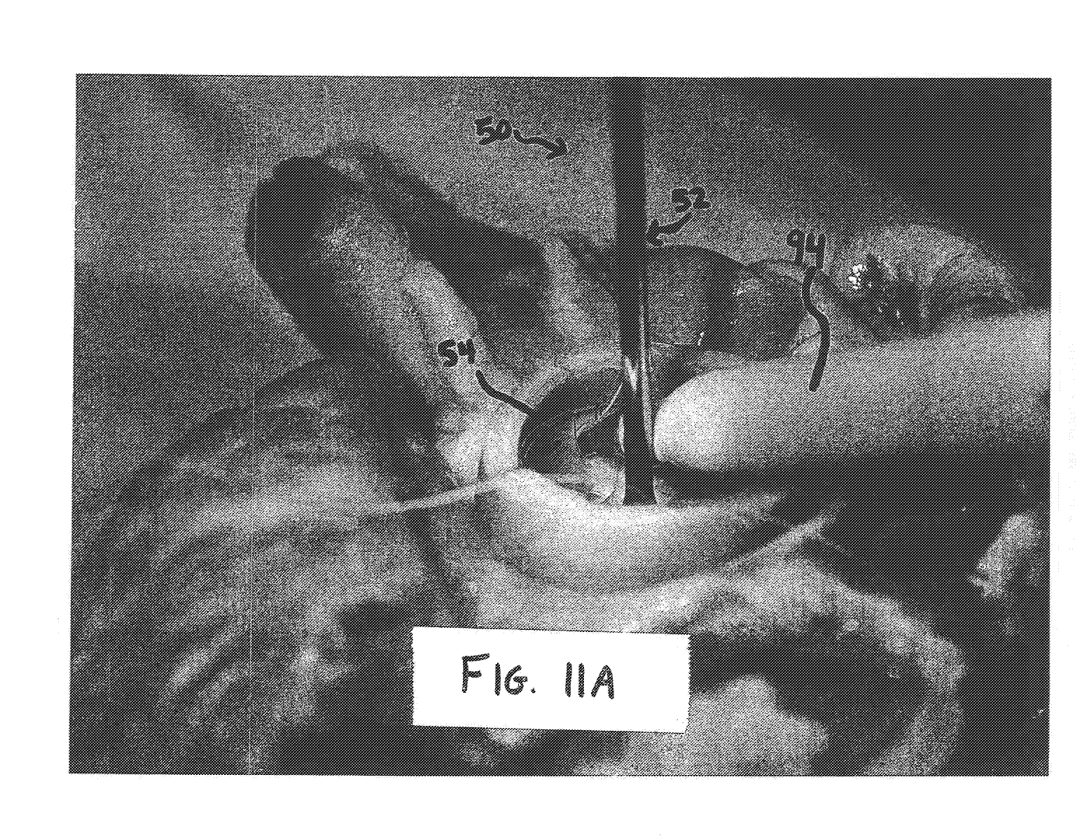

Once the lower end of each of the commissures adjacent to the inflow annulus of the aortic sinus region 90 has been marked, the positioning body 54 is then retracted as illustrated in FIG. 10 until the first end 70 is in the same plane as the inflow annulus and the alignment notches 76A, 76B, and 76C align with the sutures 92A, 92B, and 92C, respectively. When the positioning body 54 has been retracted in such a manner, the alignment notches 77A, 77B, and 77C will be positioned in the region of sinotubular junction due to the fixed distance 78 (see FIG. 7) between each of the notches in a pair as discussed above. The surgeon may then mark the current position of the alignment notches 77A, 77B, and 77C with any suitable marking means, such as with a second set of sutures similar to those used to mark the lower end of each of the commissures.

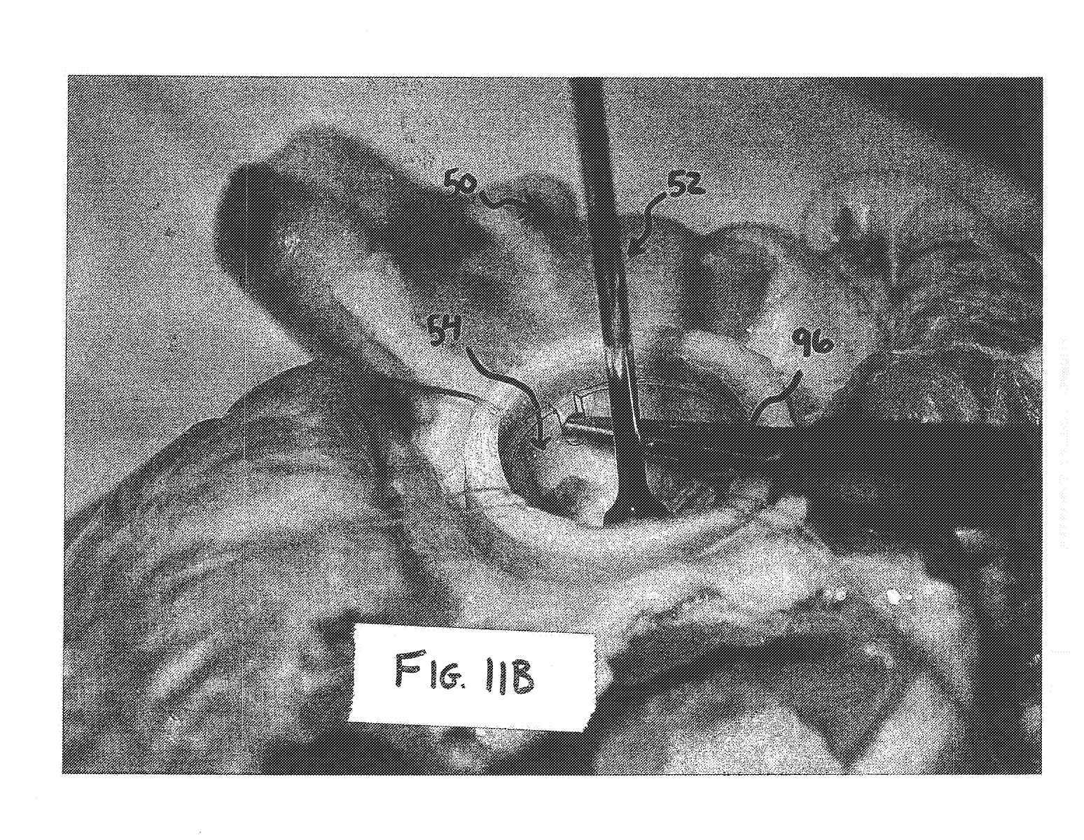

Although the marking steps have been described with reference to sutures, any suitable marking means may be used as will be appreciated by those skilled in the art. These marking means may include, but are not limited to, an ink marking pen 94 for creating an ink spot as illustrated in FIG. 11A or a cauterizing tool 96 for creating a burn mark as illustrated in FIG. 11B.

Once the surgeon has marked the positions of the three pairs of notches, these markings function as a template for proper placement of a replacement valve. The three markings in the region of sinotubular junction help to ensure that the commissural tabs of the replacement valve are aligned with the commissures and separated by about 120 degrees. The three markings adjacent to the inflow annulus of the aortic sinus region help to ensure that the inflow end of the replacement valve is positioned in the correct plane within the aortic root, and that the outflow end of the valve is not twisted (i.e., angularly misaligned) with respect to the inflow end of the valve. The three markings adjacent to the inflow annulus of the aortic sinus region also help to ensure that the commissural tabs of the replacement valve are positioned at the proper height relative to the inflow annulus.

The positioning tool 50 and its method of use were described with reference to three pairs of notches for creating three pairs of markings merely for purposes of example and not limitation. Thus, the device and method of the present invention may be adapted for use in the placement and positioning of a valve having a fewer or greater number of leaflets without departing from the intended scope of the present invention.

Although the present invention has been described with reference to preferred embodiments, workers skilled in the art will recognize that changes may be made in form and detail without departing from the spirit and scope of the invention.

What is claimed is:

1. A positioning tool comprising:
an elongate positioning member having a first end and a second end;
a tubular positioning body coupled to the first end of the elongate positioning member, the tubular positioning body including a first end, a second end, and an attachment structure disposed within an open center of the positioning body; and
a plurality of pairs of alignment apertures, each pair of alignment apertures including a first alignment aperture adjacent to the first end of the positioning body and a second alignment aperture adjacent to the second end of the positioning body.

2. The positioning tool of claim 1, wherein the first alignment apertures are formed as notches in the first end of the positioning body and the second alignment apertures are formed as notches in the second end of the positioning body.

3. The positioning tool of claim 1, wherein the positioning body is detachably coupled to the elongate positioning member.

4. The positioning tool of claim 3, wherein the positioning body is detachably coupled to the elongate positioning member with a threaded connection.

5. The positioning tool of claim 1, wherein the elongate positioning member includes a handle portion and a shaft portion.

6. The positioning tool of claim 5, wherein the handle portion includes gripping means.

7. The positioning tool of claim 1, wherein the number of pairs of alignment apertures is three.

8. The positioning tool of claim 7, wherein the three pairs of alignment apertures are spaced around a circumference of the positioning body in increments of about 120 degrees.

9. The positioning tool of claim 1, wherein the first and second alignment apertures in each pair of alignment apertures are positioned along a line that is substantially perpendicular to the first and second ends of the positioning body such that the first and second alignment apertures are angularly aligned with one another.

10. The positioning tool of claim 1, wherein a distance between the first and second alignment apertures in each pair of alignment apertures corresponds with a height of an aortic valve sinus.

11. A method for positioning a heart valve comprising:
providing a positioning tool comprising:
an elongate positioning member;
a positioning body disposed at an end of the elongate positioning member; and
a plurality of pairs of alignment apertures formed in the positioning body; each pair of alignment apertures including spaced apart first and second alignment apertures;
sliding the positioning body into an aortic sinus region until the second alignment apertures are positioned adjacent to an inflow annulus of the sinus region;
aligning each of the second alignment apertures with an end of a commissure adjacent to the inflow annulus;
marking a first commissure location of each of the second alignment apertures;
retracting the positioning body until the first alignment apertures are substantially coplanar with the inflow annulus and aligned with the marked first commissure locations; and
marking a second commissure location of each of the second alignment apertures.

12. The method of claim 11, wherein the number of pairs of alignment apertures is three.

13. The method of claim 12, wherein the three pairs of alignment apertures are spaced around a circumference of the positioning body in increments of about 120 degrees.

14. The method of claim 11, wherein the second commissure locations correspond to an end of the commissure adjacent to the region of sinotubular junction.

15. The method of claim 11, further comprising positioning a heart valve within the sinus and attaching a plurality of commissural tabs at the second commissure locations.
